(19) United States
(12) Patent Application Publication
(10) Pub. No.: US 2009/0137593 A1
(43) Pub. Date: May 28, 2009

(54) 4-AMINO-2,6-DIMETHYLPHENOL COMPOUND, PROCESS FOR ITS PREPARATION AND PHARMACEUTICAL COMPOSITION COMPRISING THE SAME

(75) Inventor: Beatrice GARRONE, Roma (IT)

Correspondence Address: OBLON, SPIVAK, MCCLELLAND MAIER & NEUSTADT, P.C., 1940 DUKE STREET, ALEXANDRIA, VA, 22314, US

(73) Assignee: AZIEN CHIM RIUNITE ANGEL FRANC A.C.R.A.F. S.P.A., Roma (IT)

(21) Appl. No.: 12/298,609

(22) PCT Filed: Mar. 30, 2007
(86) PCT No.: PCT/EP2007/002965

§ 371 Date: Nov. 28, 2008

(30) Foreign Application Priority Data
Apr. 27, 2006 (IT) MI2006A000836

(51) Int. Cl. A61K 31/5375; C07D 295/14

(52) U.S. Cl. 514/238.2; 544/165

(57) ABSTRACT

A 4-amino-2,6-dimethylphenol compound having the formula given in the description, acid-addition salts thereof, a process for preparing them, pharmaceutical composition comprising them and its use as anti-pyretic and anti-nociceptive agents.

The present invention relates to a 4-amino-2,6-dimethylphenol compound and acid-addition salts thereof, a process for preparing them and a pharmaceutical composition comprising the same.

More particularly, the 4-amino-2,6-dimethylphenol compound of present invention is the N-(4-hydroxy-3,5-dimethylphenyl)-2-morpholin-4-yl acetamide having the following formula (I):

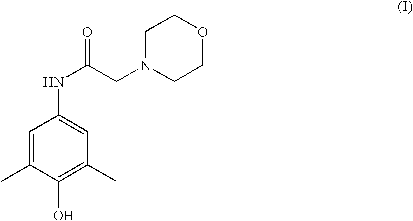

and is endowed with anti-pyretic and anti-nociceptive activity.

JP 02-160 751 and 04-041 425 relate to 5-lipoxygenase-inhibiting compounds that are useful as antiallergic and anti-inflammatory agents, of general formula (III):

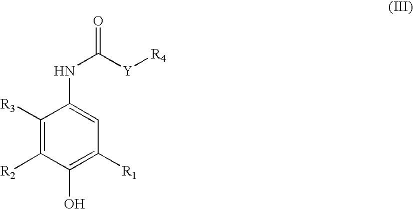

wherein:
R₁ and R₂ are lower alkyl, lower alkanoyl or cycloalkyl;
R₃ is hydrogen or lower alkyl;
Y is a single bond, lower alkenyl or lower alkenylene; and
R₄ is naphthyl, naphthyloxy or an optionally substituted heterocyclic ring, or diphenyl-(lower)alkyl.

The compound of formula (I) is thus included among the compounds of formula (III), but documents JP 02-160 751 and 04-041 425 do not specifically exemplify the same and do not describe its pharmacological properties.

The closest compound to that of formula (I) that was illustrated in the abovementioned documents is the compound of formula (II):

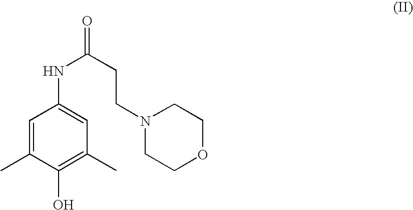

which will also be referred to hereinbelow as the Comparison Compound.

Surprisingly, it has now been found, that the compound of formula (I) and the acid-addition salts thereof are free of 5-lipoxygenase-inhibiting activity and that, however, they are endowed with anti-pyretic and anti-nociceptive activity. In particular, the compound of formula (I) and the acid-addition salts thereof proved to be free of 5-lipoxygenase-inhibiting activity in the pharmacological tests described in document JP 02-160 751.

This is all the more surprising since it has also been found that the Comparison Compound of formula (II) is free of both anti-pyretic activity and anti-nociceptive activity.

In a first aspect, the present invention therefore relates to a compound of formula (I) and the acid-addition salts thereof with physiologically acceptable inorganic and organic acids.

Typical examples of suitable physiologically acceptable inorganic acids are hydrochloric, hydrobromic, sulfuric, phosphoric and nitric acid.

Typical examples of suitable physiologically acceptable organic acids are acetic, ascorbic, oxalic, succinic, maleic, fumaric, tartaric, methane sulfonic, tannic and benzoic acid.

In a second aspect, the present invention relates to a method for preparing the compound of formula (I) and the physiologically acceptable acid-addition salts thereof, characterized in that 4-morpholineacetic acid is reacted with 4-amino-2,6-dimethylphenol according to the following scheme:

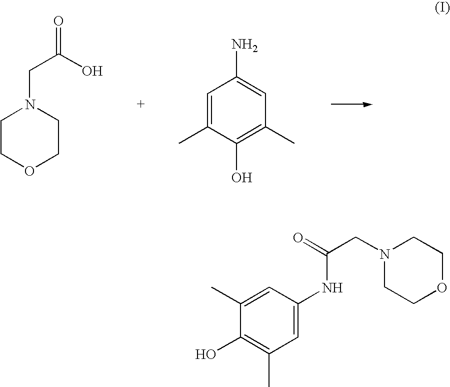

in the presence of a suitable solvent, and that the compound thus obtained is optionally converted into a physiologically acceptable acid-addition salt thereof.

Preferably, the said reaction is performed by reacting 4-morpholine-acetic acid with 4-amino-2,6-dimethylphenol in the presence of a suitable coupling agent.

A typical example of suitable coupling agents is N,N′-dicyclohexyl-carbodiimide (DCC).

Advantageously, the 4-morpholineacetic acid is reacted as hydrochloride salt and in the presence of a suitable base.

A typical example of a suitable base is 4-dimethylaminopyridine (DMAP).

Preferably, the solvent is a dipolar aprotic organic solvent, for instance dichloromethane (DCM).

The intermediate product 4-amino-2,6-dimethylphenol is preferably prepared by reduction of 4-nitro-2,6-dimethylphenol. Typically, this reduction is performed in the presence of 10% palladium-on-charcoal under a hydrogen atmosphere.

The 4-morpholineacetic acid hydrochloride, in turn, may be prepared by reacting morpholine with ethyl bromoacetate according to conventional techniques.

In a third aspect the present invention relates to a pharmaceutical composition comprising an effective dose of a compound of formula (I), or a physiologically acceptable acid-addition salt thereof, and at least one pharmaceutically acceptable inert ingredient.

Preferably, the pharmaceutical compositions of the present invention are prepared in suitable dosage forms comprising an effective dose of a compound of formula (I), or of a physiologically acceptable acid-addition salt thereof, and at least one pharmaceutically acceptable inert ingredient.

Typical examples of pathological conditions that may benefit from treatment with a pharmaceutical composition according to the present invention are febrile states and pain.

Examples of suitable dosage forms are tablets, capsules, coated tablets, granules, solutions and syrups for oral administration; medicated plasters for transdermal administration; suppositories for rectal administration and injectable sterile solutions.

Other suitable dosage forms are sustained-release forms and liposome-based forms for oral, injectable or transdermal administration.

The dosage forms may also contain other conventional ingredients such as preserving agents, stabilizers, surfactants, buffers, salts for adjusting the osmotic pressure, emulsifiers, sweeteners, colourings, flavourings, and the like.

If needed for particular therapeutic purposes, the pharmaceutical composition of the present invention may contain other pharmacologically active ingredients whose simultaneous administration is useful.

The amount of compound of formula (I), or of a physiologically acceptable acid-addition salt thereof, in the pharmaceutical composition of the present invention may vary within a wide range depending on known factors such as, for example, the type of pathology, the severity of the disease, the bodyweight of the patient, the dosage form, the selected route of administration and the number of daily administrations.

However, the optimum amount may be determined by a person skilled in the art in an easy and routine manner.

Typically, the amount of compound of formula (I), or of a physiologically acceptable acid-addition salt thereof, in the pharmaceutical composition of the present invention will be such as to ensure a level of administration of from 0.001 to 100 mg/kg/day.

This level will preferably be of from 0.05 to 50 mg/kg/day and even more preferably of from 0.1 to 10 mg/kg/day.

The dosage forms of the pharmaceutical composition of the present invention may be prepared according to techniques that are well known to pharmaceutical chemists and involve mixing, granulating, tabletting, dissolving, sterilizing and the like.

In a fourth aspect, the present invention relates to a method of treatment wherein to a patient suffering from a febrile state or pain it is administered an effective amount of a compound of formula (I), or a physiologically acceptable acid-addition salt thereof.

The following examples and tests are given to further illustrate the present invention without, however, limiting it in any way.

### EXAMPLE 1

### N-(4-Hydroxy-3,5-dimethylphenyl)-2-morpholin-4-yl acetamide

### a) Morpholin-4-yl Acetic Acid Hydrochloride

28.5 ml of ethyl bromoacetate (MW=167.0; d=1.51 g/ml; 258 mmol) were added slowly to a solution of 45 g of morpholine (MW=87; d=0.995 g/ml; 517 mmol) in 200 ml of diethyl ether. After stirring the mixture for 2 hours at room temperature, the solid formed was removed by filtration.

The solution thus obtained was concentrated under reduced pressure. The residue obtained was treated with a solution of 18 g of KOH (MW=56.1; 321 mmol) in 180 ml of ethanol for 4 hours at room temperature. The mixture was then made acid by adding a solution of 6N hydrochloric acid in ethanol (about 100 ml) and the solid product formed was then removed by filtration and washed with ethanol.

This solid was the morpholin-4-yl acetic acid hydrochloride (48 g), which was used in subsequent reactions without further purification.

LC/MS (M+H)⁺=146; 100 (base peak).

### b) 4-Amino-2,6-dimethylphenol

A suspension of 50 g of 4-nitro-2,6-dimethylphenol (MW=167.16; 294.5 mmol) and 5 g of 10% palladium-on-charcoal (MW=106.41; 4.7 mmol) in 1000 ml of ethanol was stirred at room temperature under a hydrogen atmosphere in a Parr hydrogenator for 4 hours. The mixture was then filtered under nitrogen through a layer of Celite and the solvent was evaporated off under reduced pressure.

The solid residue thus obtained was 4-amino-2,6-dimethylphenol (38 g), which was used in subsequent reactions without further purification.

LC/MS (M+H)⁺=138; 123 (base peak).

¹H-NMR (δ, DMSO d-6) 7.5-6.8 (broad s, 1H); 6.16 (s, 2H); 4.5-3.9 (broad s, 2H); 2.03 (s, 6H).

### c) N-(4-Hydroxy-3,5-dimethylphenyl)-2-morpholin-4-yl Acetamide

### Compound 1

To a suspension of 34.5 g of morpholin-4-yl acetic acid hydrochloride from Example 1a) (MW=181.6; 190 mmol) in 500 ml of dichloro-methane were added 42 g of 4-dimethylaminopyridine (MW=122.17; 344 mmol) and, after a few minutes, 71 g of N,N′-dicyclohexylcarbo-diimide (MW=206.33, 344 mmol).

23.3 g of 4-amino-2,6-dimethylphenol from Example 1b) (MW=137.18; 170 mmol) were added to the stirred mixture under a nitrogen atmosphere and at room temperature.

The mixture was stirred for 24 hours, after which the solid formed was removed by filtration. The solution was then concentrated under reduced pressure. The residue was taken up with a hexane/ethyl acetate 1:1 mixture and filtered again through a layer of silica.

The resulting solution was concentrated under reduced pressure and the residue was purified by flash chromatography on silica, using a chloroform/methanol 9:1 mixture as eluent.

The solid residue thus obtained was N-(4-hydroxy-3,5-dimethyl-phenyl)-2-morpholin-4-yl acetamide, which was crystallized twice from ethyl acetate and then used in the subsequent reactions without further purification.

### EXAMPLE 2

### N-(4-Hydroxy-3,5-dimethylphenyl)-2-morpholin-4-yl acetamide hydrochloride

### Compound 2

The N-(4-hydroxy-3,5-dimethylphenyl)-2-morpholin-4-yl acetamide obtained in Example 1c) was dissolved in an ethyl acetate/ethanol 2:1 mixture and treated with an excess of 5N hydrochloric acid in ethanol for 3 hours at room temperature.

The resulting solid was separated by filtration, crystallized from ethanol and then filtered again and dried in a vacuum oven. 10.1 g of N-(4-hydroxy-3,5-dimethylphenyl)-2-morpholin-4-yl acetamide hydrochloride were thus obtained.

m.p.=224° C. with decomposition

Elemental analysis for C₁₄H₂₀N₂O₃•HCl

| | C | H | N | Cl |
|---|---|---|---|---|
| Found % | 55.78 | 7.01 | 9.15 | 11.86 |
| Calculated % | 55.90 | 7.04 | 9.31 | 11.79 |

LC/MS (M+H)⁺=265; 100 (base peak)

¹H-NMR (δ, DMSO d-6) 11.3-10.4 (2 broad s, 2H); 8.5-7.8 (broad s, 1H); 7.17 (s, 2H); 4.2-3.1 (m, 10H); 2.14 (s, 6H).

### EXAMPLE 3

### N-(4-Hydroxy-3,5-dimethylphenyl)-3-morpholin-4-yl propanamide hydrochloride

### Comparison Compound

N-(4-Hydroxy-3,5-dimethylphenyl)-3-morpholin-4-yl propanamide was prepared, in the form of hydrochloride salt, as described in document JP 04-041 425.

### Test 1

### Effect on the Enzyme 5-LO In Vitro

Human PMNL cells (polymorphonuclear leukocytes) were used.

The cells were incubated in the presence of N-(4-hydroxy-3,5-di-methylphenyl)-2-morpholin-4-yl acetamide hydrochloride (Compound 2), using arachidonic acid as reaction substrate.

The activity of the enzyme 5-LO was evaluated by quantifying leukotriene (LT4), which is the final product of the enzymatic reaction.

The IC₅₀ of Compound 2 on the enzyme 5-LO was of 31 μM.

As is known to those skilled in the art, in this test, compounds with significant activity have an IC₅₀ value≦1 μM (for example, NDGA: IC₅₀=0.13 μM; Carter G W et al., J. Pharmacol. Exp. Ther., 1991, 256(3): 929-937).

Thus, the abovementioned IC₅₀ value of 31 μM shows that Compound 2 of the present invention is inactive with respect to the enzyme 5-LO.

### Test 2

### Effect on 5-LO-Mediated Bronchoconstriction In Vivo

Guinea pigs weighing 250-400 g were used.

The animals were sensitized by administration on days 1 and 8 of ovalbumin (0.5 μg/animal, ip). Between days 19 and 23 after the sensitization, the capacity of ovalbumin (15 μg/kg, iv) to induce bronchoconstriction was tested by measuring the increase in intratracheal pressure.

On the day of the test, 30 minutes after administration of Compound 2 (100 mg/kg, ip), the animals were treated with mepiramine (2 mg/kg, iv), indomethacin (10 mg/kg, iv) and propranolol (0.1 mg/kg, iv), and then received ovalbumin (15 μg/kg, iv) to induce bronchoconstriction. The control animals received intraperitoneally only the vehicle used (methylcellulose) for the administration of the tested compound.

The effects on 5-LO-mediated bronchoconstriction in vivo were evaluated by measuring the intratracheal pressure, and are expressed as a percentage of inhibition of bronchoconstriction.

Compound 2 induced a 7% inhibition.

As is known to those skilled in the art, in this test, compounds with significant activity have a % of inhibition>70% (for example phenidone: % inhibition=86; Sakuma Y et al., Prostaglandins, 1991, 41(4): 315-329).

Thus, the abovementioned inhibition value of 7% means that Compound 2 of the present invention is inactive with respect to inhibiting 5-LO-mediated bronchoconstriction in vivo.

### Test 3

### Effects on PAN-Induced Nephrosis

This test allows the in vivo evaluation of the effects on renal damage induced by the inflammatory response following the administration of PAN (puromycin aminonucleoside).

Male SD rats weighing 180-220 g were used.

The nephrosis was induced by intravenous injection of PAN (100 mg/kg). On the sixth day of administration of PAN, the presence of nephrosis was determined by measuring the amount of protein excreted in the urine over 24 hours (basal proteinuria). On the seventh day, the rats were treated intraperitoneally with Compound 2 (100 mg/kg) and for the next 24 hours the urine was collected to measure the proteinuria. By following the same method, the control animals received, intraperitoneally, only the vehicle (methylcellulose) used to administer the tested compound.

The effects on the PAN-induced nephrosis were evaluated by measuring the proteinuria over 24 hours and expressed as a percentage of inhibition of the amount of protein excreted.

Compound 2 induced a 2% inhibition.

As is known to those skilled in the art, in this test, compounds with significant activity show a percentage of inhibition of at least 25%.

Thus, the abovementioned inhibition value of 2% means that Compound 2 of the present invention is inactive with respect to PAN-induced nephrosis.

### Test 4

### Effects on Nephritis Induced by Anti-GBM Antibodies

This test allows evaluation in vivo of the effects on renal damage induced by the inflammatory response following the administration of anti-GBM antibodies (anti-glomerular basal membrane antibodies).

Male WKY rats weighing 180-220 g were used.

The nephritis was induced by intravenous injection of anti-GBM antibodies. Immediately after administration of the antibody and 6 hours later, Compound 2 was administered intraperitoneally (100 mg/kg). The treatment was continued for the following 9 days, with 2 administrations per day of the tested product.

By following the same methods, the control animals received only the vehicle (methylcellulose) used to administer the tested compound.

On the tenth day from injection of the antibodies, the effects on nephritis were evaluated by measuring the proteinuria over 24 hours and expressed as a percentage of inhibition of the amount of protein excreted.

Compound 2 induced a 1% inhibition.

As is known to those skilled in the art, in this test, compounds with significant activity show a percentage of inhibition of at least 25%.

Thus, the abovementioned inhibition value of 1% means that Compound 2 of the present invention is inactive with respect to nephritis induced by anti-GBM antibodies.

### Test 5

### Anti-Pyretic Activity

This test allows in vivo evaluation of the effects on the yeast-induced febrile response.

Male CD rats weighing 101-125 g were used.

Fevers were induced by subcutaneous injection of a 15% yeast suspension.

18 hours after the administration of yeast, a first group of animals was treated intraperitoneally (30 mg/kg) with Compound 2, while a second group of animals was treated with the Comparison Compound.

A third group of control animals received intraperitoneally only the vehicle (methylcellulose) used to administer the tested compounds.

For all the groups, the rectal temperature was measured 30 minutes and 1, 2 and 4 hours after the treatment.

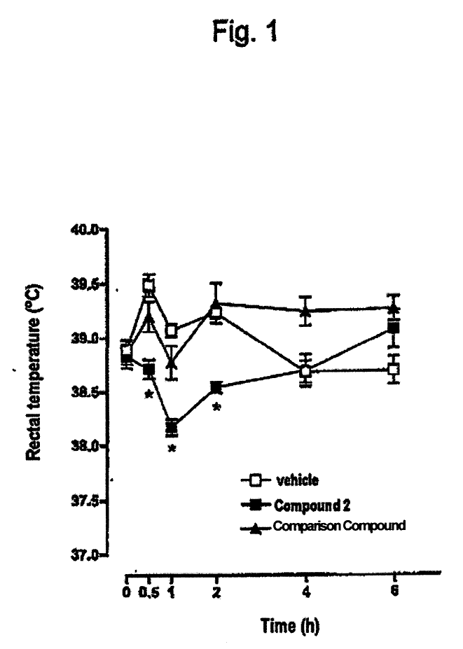

The results are shown in FIG. 1 and show that Compound 2 of the present invention induces a significant reduction in the febrile response within 1 hour of the treatment and that the effects last for about 4 hours.

In contrast, under the same experimental conditions, the Comparison Compound is incapable of reducing the febrile response.

### Test 6

### Anti-Nociceptive Activity

This test allows the in vivo evaluation of the effects on the nociceptive response induced by mechanical hyperalgesia.

Male CD rats weighing 101-125 g were used.

The hyperalgesia was induced by intraplantar injection of 1% carrageenan (100 μl/rat). The nociceptive response was evaluated by measuring the mechanical pain threshold by using an analgesimeter.

Compound 2 of the present invention and the Comparison Compound were administered intraperitoneally 2 hours after the injection of carrageenan and the nociceptive response was evaluated 1, 2 and 4 hours after the treatment. The control animals received intraperitoneally only the vehicle (methylcellulose) used to administer the tested compounds. The results, expressed as ED₅₀, are as follows:

Compound 2, ED₅₀=3 mg/kg;

Comparison Compound, ED₅₀>100 mg/kg.

Compound 2 of the present invention thus has significant anti-nociceptive activity, whereas the Comparison Compound was found to be inactive.

1. A compound of formula (I) 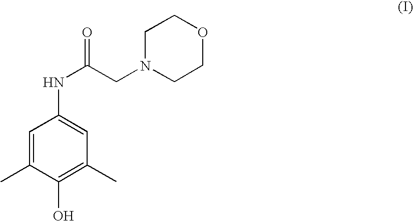
and acid-addition salts thereof with physiologically acceptable inorganic and organic acids.

2. A compound according to claim 1, characterized in that the physiologically acceptable inorganic or organic acid is selected from the group comprising hydrochloric acid, hydrobromic acid, sulfuric acid, phosphoric acid, nitric acid, acetic acid, ascorbic acid, oxalic acid, succinic acid, maleic acid, fumaric acid, tartaric acid, methanesulfonic acid, tannic acid and benzoic acid.

3. A method for preparing the compound of formula (I): 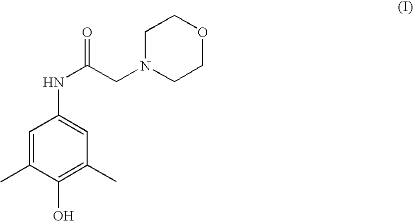
and the physiologically acceptable acid-addition salts thereof, characterized in that morpholin-4-yl acetic acid is reacted with 4-amino-2,6-dimethylphenol according to the following scheme: 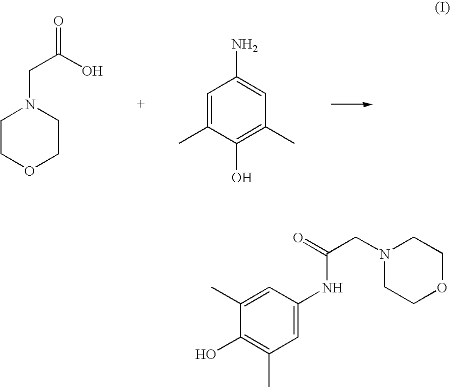
in the presence of a suitable solvent, and optionally, the compound thus obtained is converted into a physiologically acceptable acid-addition salt thereof.

4. A method according to claim 3, characterized in that morpholin-4-yl acetic acid is reacted with 4-amino-2,6-dimethylphenol in the presence of a suitable coupling agent.

5. A method according to claim 4, characterized in that the coupling agent is N,N′-dicyclohexylcarbodiimide.

6. A method according to any one of claims 3 to 5, characterized in that morpholin-4-yl acetic acid is reacted in the form of hydrochloride salt and in the presence of a suitable base.

7. A method according to claim 6, characterized in that the base is 4-dimethylaminopyridine.

8. A method according to any one of claims 3 to 7, characterized in that the solvent is a dipolar aprotic organic solvent.

9. A method according to claim 8, characterized in that the dipolar aprotic organic solvent is dichloromethane.

10. A pharmaceutical composition, characterized in that it comprises an effective dose of a compound of formula (I): 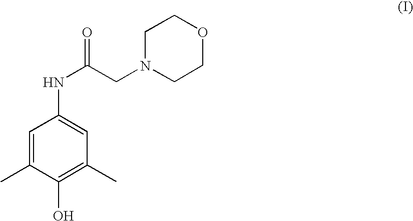
or of a physiologically acceptable acid-addition salt thereof, and at least one pharmaceutically acceptable inert ingredient.

11. A method of treatment in which an effective amount of a compound of formula (I): 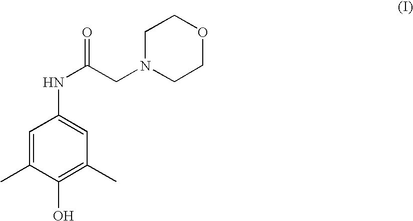
or of a physiologically acceptable acid-addition salt thereof, is administered to a patient suffering from a febrile state or pain.
